(19) United States
(12) Patent Application Publication
(10) Pub. No.: US 2024/0363213 A1
(43) Pub. Date: Oct. 31, 2024

(54) APPARATUS AND METHOD FOR AN ANTI-AGING TREATMENT

(71) Applicant: Gowri Rocco, Corona, CA (US)

(72) Inventor: Gowri Rocco, Corona, CA (US)

(21) Appl. No.: 18/139,643

(22) Filed: Apr. 26, 2023

(51) Int. Cl. G16H 20/10

(52) U.S. Cl. CPC G16H 20/10

(57) ABSTRACT

An apparatus and method for an anti-aging treatment, the apparatus including at least a processor, a memory connected to the at least a processor, the memory containing instructions configuring the at least a processor to receive user data, receive diagnostic test data from a plurality of diagnostic tests, identify a first bioidentical hormone replacement associated with the user data and diagnostic test data, identify a second bioidentical hormone replacement therapy associated with the user data and diagnostic test data and generate an anti-aging treatment as a function of the user data, the diagnostic test data, the first bioidentical hormone replacement therapy, and a second bioidentical hormone replacement therapy.

### FIELD OF THE INVENTION

The present invention generally relates to the field of anti-aging medicine. In particular, the present invention is directed to an apparatus and method for an anti-aging treatment.

### BACKGROUND

Aging is the predominant risk factor for most conditions and diseases that limit the human health span. There are various known sources of aging, these include changes in the endocrine system, such as decreasing hormone levels like estrogen in women and testosterone in men. For both men and women, these changes can have widespread effects on the deterioration of both physical and mental health. Many people believe that aging is inevitable, and an effective anti-aging treatment remains an elusive goal.

### SUMMARY OF THE DISCLOSURE

In an aspect, an apparatus for an anti-aging treatment, the apparatus including at least a processor, a memory connected to the at least a processor, the memory containing instructions configuring the at least a processor to receive user data, receive diagnostic test data from a plurality of diagnostic tests, identify a first bioidentical hormone replacement associated with the user data and diagnostic test data, identify a second bioidentical hormone replacement therapy associated with the user data and diagnostic test data and generate an anti-aging treatment as a function of the user data, the diagnostic test data, the first bioidentical hormone replacement therapy, and the second bioidentical hormone replacement therapy.

In another aspect, a method for an anti-aging treatment including receiving, by the processor, user data, receiving diagnostic test data from a plurality of diagnostic tests, identifying a first bioidentical hormone replacement associated with the user data and diagnostic test data, identifying a second bioidentical hormone replacement therapy associated with the user data and diagnostic test data and generating an anti-aging treatment as a function of the user data, the diagnostic test data, the first bioidentical hormone replacement therapy, and the second bioidentical hormone replacement therapy.

These and other aspects and features of non-limiting embodiments of the present invention will become apparent to those skilled in the art upon review of the following description of specific non-limiting embodiments of the invention in conjunction with the accompanying drawings.

### BRIEF DESCRIPTION OF THE DRAWINGS

For the purpose of illustrating the invention, the drawings show aspects of one or more embodiments of the invention. However, it should be understood that the present invention is not limited to the precise arrangements and instrumentalities shown in the drawings, wherein:

FIG. 1 is a block diagram of an exemplary embodiment of an apparatus for an anti-aging treatment;

FIG. 2 is an illustration of an exemplary embodiment of a database;

FIG. 3 is a diagram of an exemplary embodiment of a machine-learning module;

FIG. 4 is a diagram of an exemplary embodiment of a neural network;

FIG. 5 is a diagram of an exemplary embodiment of a node of a neural network;

FIG. 6 is a diagram of an exemplary embodiment of a fuzzy set comparison;

FIG. 7 is an exemplary embodiment of a method for an anti-aging treatment; and

FIG. 8 is a block diagram of a computing system that can be used to implement any one or more of the methodologies disclosed herein and any one or more portions thereof.

The drawings are not necessarily to scale and may be illustrated by phantom lines, diagrammatic representations and fragmentary views. In certain instances, details that are not necessary for an understanding of the embodiments or that render other details difficult to perceive may have been omitted.

### DETAILED DESCRIPTION

At a high level, aspects of the present disclosure are directed to apparatus and methods for an anti-aging treatment. Aspects of the present disclosure may treat hormone imbalance, sleep deprivation, muscle weakness, hair loss, memory loss, weight gain and the like.

Aspects of the present disclosure can be administered orally or by injection to counter the anti-aging process. Exemplary embodiments illustrating aspects of the present disclosure are described below in the context of several specific examples.

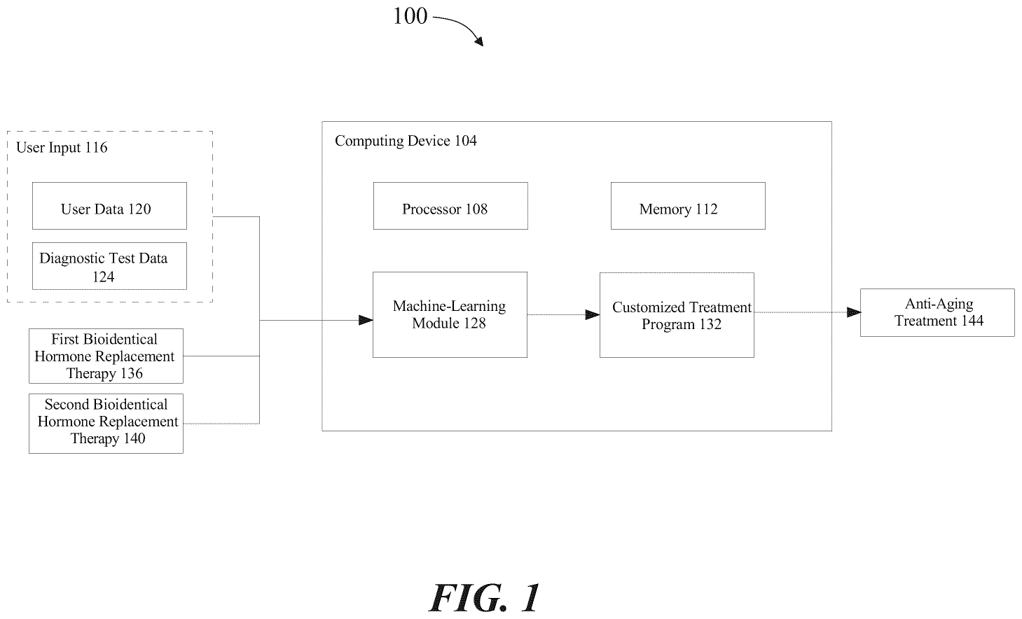

Referring now to FIG. 1, an exemplary embodiment of an apparatus 100 for an anti-aging treatment is illustrated. Apparatus 100 may include, be included in, and/or be a computing device 104. Computing device 104 may be designed and/or configured to perform any method, method step, or sequence of method steps in any embodiment described in this disclosure, in any order and with any degree of repetition. For instance, a computing device 104 may be configured to perform a single step or sequence repeatedly until a desired or commanded outcome is achieved; repetition of a step or a sequence of steps may be performed iteratively and/or recursively using outputs of previous repetitions as inputs to subsequent repetitions, aggregating inputs and/or outputs of repetitions to produce an aggregate result, reduction or decrement of one or more variables such as global variables, and/or division of a larger processing task into a set of iteratively addressed smaller processing tasks. Computing device 104 may perform any step or sequence of steps as described in this disclosure in parallel, such as simultaneously and/or substantially simultaneously performing a step two or more times using two or more parallel threads, processor cores, or the like; division of tasks between parallel threads and/or processes may be performed according to any protocol suitable for division of tasks between iterations. Persons skilled in the art, upon reviewing the entirety of this disclosure, will be aware of various ways in which steps, sequences of steps, processing tasks, and/or data may be subdivided, shared, or otherwise dealt with using iteration, recursion, and/or parallel processing.

Apparatus 100 also includes a processor 108. Processor 108 may include any processor incorporated in any computing device as described in this disclosure, including without limitation a microcontroller, microprocessor, digital signal processor (DSP) and/or system on a chip (SoC) as described in this disclosure. Processor and/or computing device may include, be included in, and/or communicate with a mobile device such as a mobile telephone or smartphone. A computing device incorporating processor 108 may include a single computing device operating independently, or may include two or more computing device operating in concert, in parallel, sequentially or the like; two or more computing devices may be included together in a single computing device or in two or more computing devices. Processor 108 and/or computing device may interface or communicate with one or more additional devices as described below in further detail via a network interface device. Network interface device may be utilized for connecting processor 108 and/or computing device to one or more of a variety of networks, and one or more devices. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network may employ a wired and/or a wireless mode of communication. In general, any network topology may be used. Information (e.g., data, software etc.) may be communicated to and/or from a computer and/or a computing device. A computing device including processor 108 may include but is not limited to, for example, a computing device or cluster of computing devices in a first location and a second computing device or cluster of computing devices in a second location. A computing device including processor 108 may include one or more computing devices dedicated to data storage, security, distribution of traffic for load balancing, and the like. A computing device including processor 108 may distribute one or more computing tasks as described below across a plurality of computing devices of computing device, which may operate in parallel, in series, redundantly, or in any other manner used for distribution of tasks or memory between computing devices. A computing device including processor 108 may be implemented using a “shared nothing” architecture in which data is cached at the worker, in an embodiment, this may enable scalability of apparatus 100 and/or computing device.

With continued reference to FIG. 1, processor 108 and/or computing device may be designed and/or configured to perform any method, method step, or sequence of method steps in any embodiment described in this disclosure, in any order and with any degree of repetition. For instance, processor 108 and/or computing device may be configured to perform a single step or sequence repeatedly until a desired or commanded outcome is achieved; repetition of a step or a sequence of steps may be performed iteratively and/or recursively using outputs of previous repetitions as inputs to subsequent repetitions, aggregating inputs and/or outputs of repetitions to produce an aggregate result, reduction or decrement of one or more variables such as global variables, and/or division of a larger processing task into a set of iteratively addressed smaller processing tasks. Processor 108 and/or computing device may perform any step or sequence of steps as described in this disclosure in parallel, such as simultaneously and/or substantially simultaneously performing a step two or more times using two or more parallel threads, processor cores, or the like; division of tasks between parallel threads and/or processes may be performed according to any protocol suitable for division of tasks between iterations. Persons skilled in the art, upon reviewing the entirety of this disclosure, will be aware of various ways in which steps, sequences of steps, processing tasks, and/or data may be subdivided, shared, or otherwise dealt with using iteration, recursion, and/or parallel processing.

Continuing to reference FIG. 1, apparatus 100 includes a memory 112, which may be implemented in any manner suitable for a primary and/or secondary memory described in this disclosure. Memory 112 may include instructions configuring processor 108 to perform various tasks. In some embodiments, apparatus 100 may include a computing device 104, where computing device includes processor 108 and/or memory 112. Memory 112 may be communicatively connected to processor 108. As used in this disclosure, “communicatively connected” means connected by way of a connection, attachment or linkage between two or more relata which allows for reception and/or transmittance of information therebetween. For example, and without limitation, this connection may be wired or wireless, direct or indirect, and between two or more components, circuits, devices, systems, and the like, which allows for reception and/or transmittance of data and/or signal(s) therebetween. Data and/or signals therebetween may include, without limitation, electrical, electromagnetic, magnetic, video, audio, radio and microwave data and/or signals, combinations thereof, and the like, among others. A communicative connection may be achieved, for example and without limitation, through wired or wireless electronic, digital or analog, communication, either directly or by way of one or more intervening devices or components. Further, communicative connection may include electrically coupling or connecting at least an output of one device, component, or circuit to at least an input of another device, component, or circuit. For example, and without limitation, via a bus or other facility for intercommunication between elements of a computing device. Communicative connecting may also include indirect connections via, for example and without limitation, wireless connection, radio communication, low power wide area network, optical communication, magnetic, capacitive, or optical coupling, and the like. In some instances, the terminology “communicatively coupled” may be used in place of communicatively connected in this disclosure.

Still referring to FIG. 1, apparatus 100 is configured to receive a user input 116. In some embodiments, apparatus 100 may receive user input 116 from one or more external computing devices. An “external computing device” as used in this disclosure is defined as any a computing device that is distinct from apparatus 100 and/or computing device. An external computing device may include any computing device as described in this disclosure. A “user input,” as used in this disclosure, is a form of data entry received from an individual and/or group of individuals, such as an individual and/or group of individuals that is using and/or interacting with apparatus 100. User input 116 may include, but is not limited to, user data 120. “User data” as used in this disclosure is defined as information related to the patient intake process such as patient social data, patient clinical data, payment plan information, Health Insurance Portability and Accountability Act (HIPAA) agreement data and the like. “Patient social data” as used in this disclosure is defined as patient information related to location or biographical data. “Patient clinical data” as used in this disclosure is defined as information gathered for the broad purpose of clinical research on the micro-level (patient care) to the macro-level (broad applications within a health system).

Still referring to FIG. 1, user input 116 may include but is not limited to diagnostic test data 124 from a plurality of diagnostic tests. “Diagnostic test data” as used in this disclosure is defined as information resulting from a medical treatment that a patient has received. In some embodiments, the plurality of tests may be conducted to gain comprehensive understanding of patient's health and customize individual treatment. “Diagnostic test” as used in this disclosure, is defined as a medical procedure performed to detect, diagnose, or monitor disease, disease processes, susceptibility or to determine a course of treatment. As a non-limiting example, the plurality of tests (e.g., medical tests) may include a complete blood count (CBC) test, kidney test, liver test, thyroid panel, urinalysis, lipid panel, growth hormone (GH) test, prolactin test, methylenetetrahydrofolate reductase (MTHFR) mutation test, insulin-like growth factor-1 (IFG-1) test, hemoglobin A1c (HbA1c) test, vitamin D3 test, comprehensive metabolic panel (CMP) test, high-sensitivity C-reactive protein (hs-CRP) test, homocysteine test, cancer screening, telomere test, lipoprotein particle test and the like. In some embodiments, the plurality of tests may include autoimmune test. An “autoimmune test” as used in this disclosure is defined as a test that screens for antinuclear antibodies, which are a category of antibodies that attach the healthy proteins within the cell nucleus. The autoimmune test may include an antinuclear antibody test (ANA) which is one of the first tests that are typically used when a patient may be showing symptoms of an autoimmune disorder. As a further non-limiting example, an autoimmune test may include gastrointestinal (GI) effect test, celiac panel, thyroid panel, cortisol urine test, diurnal cortisol test and the like. In some embodiments, the plurality of tests may include toxicology panel. As a non-limiting example, the toxicology panel may include stool test, drug test, serum toxicology test, hair follicle test and the like. In some embodiments, the plurality of tests may include genetic testing. “Genetic test” as used in this disclosure is defined as a test which is used to identify changes in DNA sequence or chromosome structure. Genetic testing may also include measuring the results of genetic changes, such as RNA analysis as an output of gene expression, or through biochemical analysis to measure specific protein output. As a non-limiting example, the genetic testing may include an epigenetic test. In some embodiments, the plurality of tests may include cancer screening tests. “Cancer screening tests” as used in this disclosure are defined as checking a patient's body for cancer before symptoms. Cancer screening tests may include blood tests, urines tests, DNA tests, medical imaging and the like. In some embodiments, the plurality of tests may include telomere testing. Telomeres protect the ends of DNA like the plastic tubes on the ends of shoelaces, and they usually shorten with aging. Made up of repetitive sequences of DNA, normal telomeres have enough length to withstand the erosion that occurs over the normal lifespan of a cell. Cells with very short telomeres may blow through these endcaps more quickly and this can lead to specific diseases Telomere testing may be performed by the flowFISH test which stands for flow cytometry and fluorescence in situ hybridization and the test measures the telomere length in each cell within a patient's blood sample. In flowFISH, scientists poke very tiny holes in the nucleus of each cell and slip in fluorescently labeled DNA that attach to telomeres. Then, scientists use a method called flow cytometry to identify the types of cells in each blood sample and quantify the fluorescent signal in cells one at a time. In some embodiments, the plurality of tests may include the TruAge test. The “TruAge test” as used in this disclosure is defined as an epigenetic test able to analyze the changes in a patient's DNA and to determine how these changes affect the patient's body and health. The test uses markers on patient's DNA called “methylation” to predict patient's biological age. Ideally, the biological age will be less than chronological age. In some embodiments, the plurality of tests may include an insulin test. An “insulin test” as used in this disclosure is defined as a test that measure the amount of insulin, the hormone that lets cells take in glucose. Glucose levels in the blood rise after meals and trigger the pancreas to make insulin and release it into the blood. As a non-limiting example, an insulin test may include fasting insulin. In some embodiments, the plurality of test may include a lipoprotein blood test. A “lipoprotein blood test” as used in this this disclosure is defined as a test which measures a patient's level of lipoprotein (a) in their blood, a high level may mean that patient is at risk for heart disease.

Still referring to FIG. 1, in some embodiments, the plurality of tests may include examining the patient's previous prescriptions, current prescriptions, family medical history, nutraceutical intake, and the like. In an embodiment, processor 108 may obtain patient information from a hospital database, an application programming interface and the like. In some embodiments, the plurality of diagnostic tests may include a plurality of male patient tests if the patient is male, and the plurality of diagnostic tests may include a plurality of female patient tests if the patient is female. For example, this may be because males may fare better in some aspects of aging while females may do better in other aspects. For example, male skin is less susceptible to signs of aging. By way of another example, females generally gray later than males. As a non-limiting example, the plurality of testing for females may include a cancer antigen 125 (CA125) test, estradiol test, progesterone test, testosterone panel, follicle stimulating hormone (FSH) test, luteinizing hormone (LH) test, dehydroepiandrosterone sulfate (DHEA) test, human chorionic gonadotropin (HCG) test, dihydrotestosterone (DHT) test, anti mullerian hormone (AMH) test, sex hormone binding globulin (SHBG) test and the like. As another non-limiting example, the plurality of testing for male may include prostate specific antigen (PSA) test, testosterone test, chemistry panel, DHEA test, DHT test, SSHBG test dehydroepiandrosterone sulfate (DHEA) test, dihydrotestosterone (DHT) test, sex hormone binding globulin (SHBG) test and the like.

Still referring to FIG. 1, apparatus 100 is configured to generate a first bioidentical hormone replacement therapy (BHRT) 136 associated with user data 120 and diagnostic test data 124. “Hormones” as used in this disclosure are defined as regulatory substances produced in an organism and transported in tissue fluids such as blood to stimulate specific cells or tissues into action. “Bioidentical hormones” as used in this disclosure are defined as compounds produced to be chemically and biologically the same as naturally occurring hormones such as estradiol and progesterone. “Bioidentical Hormone Replacement Therapy” as used in this disclosure is defined as the use of hormones that are identical on a molecular level with endogenous hormones in hormone replacement therapy. Bioidentical hormone replacement therapy (BHRT) uses hormones chemically identical to those produced naturally in the body and may assist in replacement, weight loss, and strength gains, as opposed to traditional hormone replacement therapy (HRT) made from the urine of pregnant horses and other synthetic hormones. Levels of most hormones decrease with aging, but some hormones remain at levels typical of those in younger adults, and some even increase. Even when hormone levels do not decline, endocrine function generally declines with age because hormone receptors become less sensitive. Some hormones that decrease with aging may include estrogen (in women), testosterone (in men), growth hormone, melatonin and the like. In women, estrogen levels may decline with menopause. In men, testosterone levels usually decrease gradually. Decreased levels of growth hormone may lead to decreased muscle mass and strength. Decreased melatonin levels may play an important role in the loss of normal sleep-wake cycles (circadian rhythms) with aging. Hormones that usually remain unchanged or only slightly decrease include cortisol, insulin, thyroid hormones. Hormones that may increase include follicle-stimulating hormone, luteinizing hormone, parathyroid hormone. Some age-related hormonal changes may impact quality of life or cause bothersome symptoms (e.g., hot flashes). In some embodiments, the BHRT may include a customized treatment program. As a non-limiting example, the customized treatment program may include customized peptides, customized growth hormones, and the like. For example, sermorelin is one of the most commonly used anti-aging peptides and may lead to an increase in muscle mass and a reduction in body fat, as well as producing firmer and tighter skin. By way of another example, injections of growth hormone may have various anti-aging properties such as increase exercise capacity, increase bone density, increase muscle mass, decrease body fat and the like.

In some embodiments, the BHRT may differ for females and males. As a non-limiting example, the BHRT for females may include topical optimized hormones, compounded thyroids prescriptions, customized nutraceuticals, lifestyle modifications, and the like. As a non-limiting example, the BHRT for males may include optimized topical hormones, compounded thyroids prescriptions, customized prescriptions, lifestyle modifications, and the like. In some embodiments, a follow up visit may be performed in the range of 3.5 to 4 weeks after the first BHRT. A “follow up visit” as used in this disclosure is defined as a visit made as a follow-up to an initial visit. For example, a follow up visit may involve blood tests, imaging tests, a physical exam and the like. The processor 108 generates the first bioidentical hormone replacement therapy (BHRT) by obtaining and analyzing user data from a database. For example, processor may access a database containing results of a patient's hormone levels, which may include diagnostic test data 124, using urine, blood, saliva tests and the like to determine which hormones may be needed to balance patient's hormone deficiency. The processor 108 may obtain the baseline/normal hormone levels from a medical index database and compare it to patient's current hormone levels.

Still referring to FIG. 1, apparatus 100 may be configured to generate a customized treatment program 132 utilizing a machine learning module 128. Training data may include a database of user data, diagnostic test data from a plurality of tests correlated to a customized treatment plan. Machine-learning module may perform determinations, classification, and/or analysis steps, methods, processes, or the like as described in this disclosure using machine learning processes. A “machine learning process,” as used in this disclosure, is a process that automatedly uses training data to generate an algorithm that will be performed by a computing device/module to produce outputs given data provided as inputs; this is in contrast to a non-machine learning software program where the commands to be executed are determined in advance by a user and written in a programming language. In one or more embodiments, a machine-learning module may be generated using training data. Training data may include inputs and corresponding predetermined outputs so that machine-learning module may use the correlations between the provided exemplary inputs and outputs to develop an algorithm and/or relationship that then allows machine-learning module to determine its own outputs for inputs. Training data may contain correlations that a machine-learning process may use to model relationships between two or more categories of data elements. The exemplary inputs and outputs may come from a database, such as any database described in this disclosure. For example, training data inputs may be patient input data and outputs may be a customized treatment program. In other embodiments, machine-learning module may obtain a training set by querying a communicatively connected database that includes past inputs and outputs. Training data may include inputs from various types of databases, resources, and/or user inputs and outputs correlated to each of those inputs so that a machine-learning module may determine an output. Correlations may indicate causative and/or predictive links between data, which may be modeled as relationships, such as mathematical relationships, by machine-learning processes, as described in further detail below. In one or more embodiments, training data may be formatted and/or organized by categories of data elements by, for example, associating data elements with one or more descriptors corresponding to categories of data elements. As a non-limiting example, training data may include data entered in standardized forms by persons or processes, such that entry of a given data element in a given field in a form may be mapped to one or more descriptors of categories. Data may include previous outputs such that the retrained machine-learning module 128 iteratively produces outputs, thus creating a feedback loop. The machine learning process is described in more detail below.

Still referring to FIG. 1, apparatus 100 is configured to generate a second bioidentical hormone replacement therapy 140 associated with user data, diagnostic test data and first bioidentical hormone replacement therapy. In some embodiments, second bioidentical hormone replacement therapy (BHRT) 140 may be titrated according to the response from the first bioidentical hormone replace therapy 136. “Titration” as used in this disclosure is defined as a technique where a solution of a known concentration is used to determine the concentration of an unknown solution. Typically the titrant (known solution) is added from a burette to a known quantity of the analyte (unknown solution) until the reaction is complete. For example, the processor 108 may receive the patient response such as patient's clinical and laboratory results according to the first bioidentical hormone replacement therapy 136. For example, patient response may include a changed hormone level. Based on the results, it may be advantageous for the processor 108 to titrate or modify the first BHRT. A processor 108 may receive information that such as patient's age, gender, lifestyle, stress level and severity of symptoms and information that patient's symptoms have not been alleviated. Processor 108 may then titrate or modify the first BHRT to a slightly higher dose in order to alleviate patient's symptoms. Processor 108 may be configured to titrate or modify the first BHRT to a higher dose by comparing the original first BHRT to a stored value and identifying the appropriate titration. For example, if patient presents with complaints of low energy levels processor 108 may receive patient's laboratory blood results which show a low level of testosterone and processor 108 may titrate the first BHRT to a slightly higher dose in order to increase patient's energy levels. For example, processor may obtain the standard/normal testosterone hormone level for patient based on patient input and compare it to patient's actual testosterone hormone level considering the first BHRT, based on this information, the processor 108 may titrate the first BHRT in order to optimize patient's testosterone level. In some embodiments, the second BHRT 140 may include addressing patient insulin resistance. “Insulin resistance” as used in this disclose is defined as an impaired response of the body to the hormone insulin, resulting in elevated levels of glucose in the blood, a key component of type 2 diabetes and metabolic syndrome. For example, there may be various types of insulin resistance syndrome such as type A insulin resistance syndrome which may be due to an absent or dysfunctional receptor, and type B insulin resistance syndrome which may result from autoantibodies to the insulin receptor. For example, patient response may show fatigue symptoms after taking the first BHRT. Processor 108 may receive patient laboratory test results indicating patient has a fasting glucose level over 100 mg/dL and a fasting triglyceride level over 150 mg/dL which indicates insulin resistance. Processor 108 may titrate the first BHRT to increase the dose of insulin in order to alleviate patient's symptoms.

Still referring to FIG. 1, the second bioidentical hormone replacement therapy 144 (BHRT) may include a target. “Target” as used in this disclosure is defined as an indicator established to determine how successful a patient is in achieving an objective or goal. As a non-limiting example, the target may include gut health, better sleep, libido, fatigue, memory improvement, hair loss, weight loss, or the like. As a non-limiting example, the second BHRT may include customized peptides for the target such as the weight loss. “Peptides” as used in this disclosure are defined as short chains of amino acids linked by peptide bonds. Peptides may initiate human growth hormone production and may break down visceral fat which may cause weight loss. For example, MOTS-c is a mitochondrial-derived peptide and may be an exercise mimetic and may provide benefits such as the regulation of obesity and physical performance. As another non-limiting example, the second BHRT may include Human Chorionic Gonadotropin (HCG) protocol and customized peptides for the target such as a clinical obesity. “Human Chorionic Gonadotropin” as used in this disclosure is defined as a hormone which is produced by the placenta during pregnancy. For example, the HCG protocol may include a human chorionic gonadotropin (HCG) hormone supplement and the restriction of calorie intake to approximately 500 calories per day to aid in weight loss.

Referring to FIG. 1, user input 116 may include, but is not limited to text input, engagement with icons of a graphical user interface (GUI), and the like. Text input may include, without limitation, entry of characters, words, strings, symbols, and the like. In some embodiments, user input 116 may include one or more interactions with one or more elements of a graphical user interface (GUI). A “graphical user interface” as used in this disclosure is an interface including set of one or more pictorial and/or graphical icons corresponding to one or more computer actions. GUI may be configured to receive user input 116. GUI may include one or more event handlers. An “event handler” as used in this disclosure is a callback routine that operates asynchronously once an event takes place. Event handlers may include, without limitation, one or more programs to perform one or more actions based on user input, such as generating pop-up windows, submitting forms, changing background colors of a webpage, and the like. Event handlers may be programmed for specific user input, such as, but not limited to, mouse clicks, mouse hovering, touchscreen input, keystrokes, and the like. For instance, and without limitation, an event handler may be programmed to generate a pop-up window if a user double clicks on a specific icon. User input 116 may include, a manipulation of computer icons, such as, but not limited to, clicking, selecting, dragging and dropping, scrolling, and the like. In some embodiments, user input 116 may include an entry of characters and/or symbols in a user input field. A “user input field” as used in this disclosure is a portion of graphical user interface configured to receive data from an individual. A user input field may include, but is not limited to, text boxes, search fields, filtering fields, and the like. In some embodiments, user input 116 may include touch input. Touch input may include, but is not limited to, single taps, double taps, triple taps, long presses, swiping gestures, and the like. In some embodiments, GUI may be displayed on, without limitation, monitors, smartphones, tablets, vehicle displays, and the like. Vehicle displays may include, without limitation, monitors and/or systems in a vehicle such as multimedia centers, digital cockpits, entertainment systems, and the like. One of ordinary skill in the art upon reading this disclosure will appreciate the various ways a user may interact with graphical user interface.

Continuing to reference FIG. 1, apparatus 100 may be configured to generate an anti-aging treatment 144. “Anti-aging treatment” as used in this disclosure is defined as a treatment using to delay or stop the aging process. The anti-aging treatment 144 may consist of prescription medicines, over the counter medications, nutraceuticals, a physical exercise program and the like. The processor 108 is configured to generate an anti-aging treatment 144 by using user data 120 and diagnostic test data 124 and combining the first BHRT 136 and second BHRT and the customized treatment program. The first and second BHRTs may be analyzed based on patient's response which may include patient's laboratory test results showing their hormone levels. These results in conjunction with the customized treatment plan 132, which may include a topical hormonal treatment and/or customized peptides, customized growth hormones, will result in the generation of the anti-aging treatment 144.

In some embodiments, the generation of the anti-aging treatment 144 includes updated diagnostic test data. The updated diagnostic test data may include a complete blood count (CBC) test, kidney test, liver test, thyroid panel, urinalysis, lipid panel, growth hormone (GH) test, prolactin test, methylenetetrahydrofolate reductase (MTHFR) mutation test, insulin-like growth factor-1 (IFG-1) test, hemoglobin A1c (HbA1c) test, vitamin D3 test, comprehensive metabolic panel (CMP) test, high-sensitivity C-reactive protein (hs-CRP) test, homocysteine test, cancer screening, telomere test, lipoprotein particle test and the like. “Updated diagnostic test data” as used in this disclosure is defined as the diagnostic test data of the user after the administration of the first and second BHRTs. In some embodiments, an additional diagnostic retest may be performed in a range of 6 to 8 weeks. The processor 108 may be configured to generate an anti-aging treatment 144 by using user data 120 and the updated diagnostic test data and combining the first BHRT 136 and second BHRT and the updated customized treatment program (based on the updated diagnostic test data). The first and second BHRTs may be analyzed based on patient's response which may include patient's laboratory test results showing their hormone levels. These results in conjunction with the updated customized treatment plan (based on the updated diagnostic test data) will result in the generation of the anti-aging treatment 144. The generation of the anti-aging treatment 144 may be continuously updated by utilizing updated diagnostic test data.

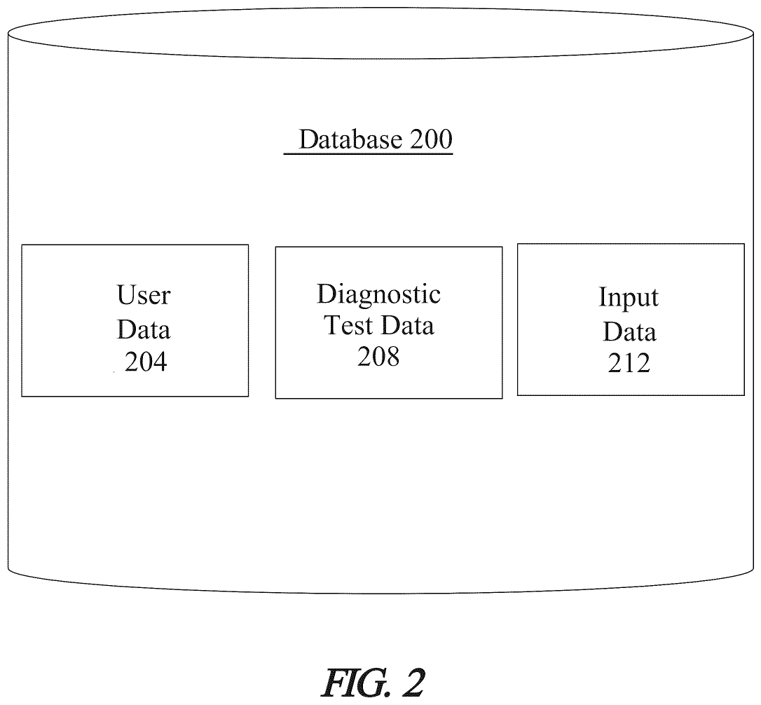

Now referencing FIG. 2, an illustration of an exemplary embodiment of a database 200 is presented. Database may be implemented, without limitation, as a relational database, a key-value retrieval database such as a NOSQL database, or any other format or structure for use as a database that a person skilled in the art would recognize as suitable upon review of the entirety of this disclosure. Database may alternatively or additionally be implemented using a distributed data storage protocol and/or data structure, such as a distributed hash table or the like. Database may include a plurality of data entries and/or records as described above. Data entries in a database may be flagged with or linked to one or more additional elements of information, which may be reflected in data entry cells and/or in linked tables such as tables related by one or more indices in a relational database. Persons skilled in the art, upon reviewing the entirety of this disclosure, will be aware of various ways in which data entries in a database may store, retrieve, organize, and/or reflect data and/or records as used herein, as well as categories and/or populations of data consistently with this disclosure.

Still referring to FIG. 2, in some embodiments, database 200 may include user data 204. User data 204 may include, without limitation, social and clinical user data. Database 200 may also include diagnostic test data 208. Diagnostic test data 208 may include, without limitation, a blood test, imaging test, physical test and the like. Database 200 may also include user input data 212. User input data 212 may include user's name, address and the like. Any and all determinations described above may be performed and analyzed using an optimization program.

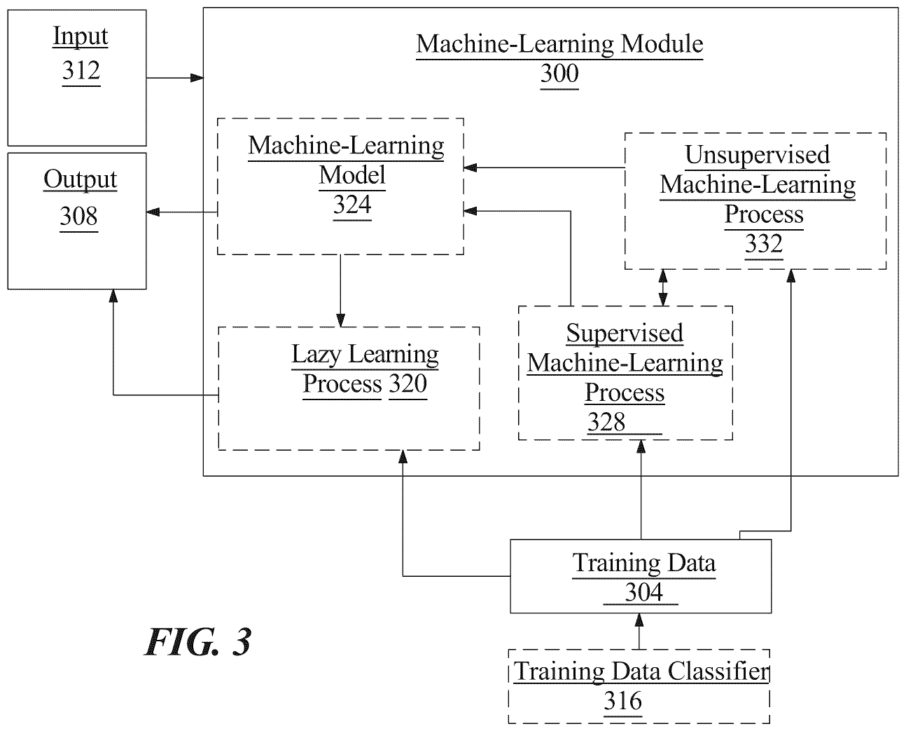

Referring now to FIG. 3, a diagram of an exemplary embodiment of a machine-learning module is presented. Machine-learning module may perform determinations, classification, and/or analysis steps, methods, processes, or the like as described in this disclosure using machine learning processes. A “machine learning process,” as used in this disclosure, is a process that automatedly uses training data 304 to generate an algorithm that will be performed by a computing device/module to produce outputs 308 given data provided as inputs 312; this is in contrast to a non-machine learning software program where the commands to be executed are determined in advance by a user and written in a programming language.

Still referring to FIG. 3, “training data,” as used herein, is data containing correlations that a machine-learning process may use to model relationships between two or more categories of data elements. For instance, and without limitation, training data 304 may include a plurality of data entries, each entry representing a set of data elements that were recorded, received, and/or generated together; data elements may be correlated by shared existence in a given data entry, by proximity in a given data entry, or the like. Multiple data entries in training data 304 may evince one or more trends in correlations between categories of data elements; for instance, and without limitation, a higher value of a first data element belonging to a first category of data element may tend to correlate to a higher value of a second data element belonging to a second category of data element, indicating a possible proportional or other mathematical relationship linking values belonging to the two categories. Multiple categories of data elements may be related in training data 304 according to various correlations; correlations may indicate causative and/or predictive links between categories of data elements, which may be modeled as relationships such as mathematical relationships by machine-learning processes as described in further detail below. Training data 304 may be formatted and/or organized by categories of data elements, for instance by associating data elements with one or more descriptors corresponding to categories of data elements. As a non-limiting example, training data 304 may include data entered in standardized forms by persons or processes, such that entry of a given data element in a given field in a form may be mapped to one or more descriptors of categories. Elements in training data 304 may be linked to descriptors of categories by tags, tokens, or other data elements; for instance, and without limitation, training data 304 may be provided in fixed-length formats, formats linking positions of data to categories such as comma-separated value (CSV) formats and/or self-describing formats such as extensible markup language (XML), JavaScript Object Notation (JSON), or the like, enabling processes or devices to detect categories of data.

Alternatively or additionally, and continuing to refer to FIG. 3, training data 304 may include one or more elements that are not categorized; that is, training data 304 may not be formatted or contain descriptors for some elements of data. Machine-learning algorithms and/or other processes may sort training data 304 according to one or more categorizations using, for instance, natural language processing algorithms, tokenization, detection of correlated values in raw data and the like; categories may be generated using correlation and/or other processing algorithms. As a non-limiting example, in a corpus of text, phrases making up a number “n” of compound words, such as nouns modified by other nouns, may be identified according to a statistically significant prevalence of n-grams containing such words in a particular order; such an n-gram may be categorized as an element of language such as a “word” to be tracked similarly to single words, generating a new category as a result of statistical analysis. Similarly, in a data entry including some textual data, a person's name may be identified by reference to a list, dictionary, or other compendium of terms, permitting ad-hoc categorization by machine-learning algorithms, and/or automated association of data in the data entry with descriptors or into a given format. The ability to categorize data entries automatedly may enable the same training data 304 to be made applicable for two or more distinct machine-learning algorithms as described in further detail below. Training data 304 used by machine-learning module 300 may correlate any input data as described in this disclosure to any output data as described in this disclosure.

Further referring to FIG. 3, training data may be filtered, sorted, and/or selected using one or more supervised and/or unsupervised machine-learning processes and/or models as described in further detail below; such models may include without limitation a training data classifier 316. Training data classifier 316 may include a “classifier,” which as used in this disclosure is a machine-learning model as defined below, such as a mathematical model, neural net, or program generated by a machine learning algorithm known as a “classification algorithm,” as described in further detail below, that sorts inputs into categories or bins of data, outputting the categories or bins of data and/or labels associated therewith. A classifier may be configured to output at least a datum that labels or otherwise identifies a set of data that are clustered together, found to be close under a distance metric as described below, or the like. Machine-learning module 300 may generate a classifier using a classification algorithm, defined as a process whereby a computing device and/or any module and/or component operating thereon derives a classifier from training data 304. Classification may be performed using, without limitation, linear classifiers such as without limitation logistic regression and/or naive Bayes classifiers, nearest neighbor classifiers such as k-nearest neighbors' classifiers, support vector machines, least squares support vector machines, fisher's linear discriminant, quadratic classifiers, decision trees, boosted trees, random forest classifiers, learning vector quantization, and/or neural network-based classifiers.

Still referring to FIG. 3, machine-learning module 300 may be configured to perform a lazy-learning process 320 and/or protocol, which may alternatively be referred to as a “lazy loading” or “call-when-needed” process and/or protocol, may be a process whereby machine learning is conducted upon receipt of an input to be converted to an output, by combining the input and training set to derive the algorithm to be used to produce the output on demand. For instance, an initial set of simulations may be performed to cover an initial heuristic and/or “first guess” at an output and/or relationship. As a non-limiting example, an initial heuristic may include a ranking of associations between inputs and elements of training data 304. Heuristic may include selecting some number of highest-ranking associations and/or training data 304 elements. Lazy learning may implement any suitable lazy learning algorithm, including without limitation a K-nearest neighbors algorithm, a lazy naïve Bayes algorithm, or the like; persons skilled in the art, upon reviewing the entirety of this disclosure, will be aware of various lazy-learning algorithms that may be applied to generate outputs as described in this disclosure, including without limitation lazy learning applications of machine-learning algorithms as described in further detail below.

Alternatively or additionally, and with continued reference to FIG. 3, machine-learning processes as described in this disclosure may be used to generate machine-learning models 324. A “machine-learning model,” as used in this disclosure, is a mathematical and/or algorithmic representation of a relationship between inputs and outputs, as generated using any machine-learning process including without limitation any process as described above and stored in memory; an input is submitted to a machine-learning model 324 once created, which generates an output based on the relationship that was derived. For instance, and without limitation, a linear regression model, generated using a linear regression algorithm, may compute a linear combination of input data using coefficients derived during machine-learning processes to calculate an output datum. As a further non-limiting example, a machine-learning model 324 may be generated by creating an artificial neural network, such as a convolutional neural network comprising an input layer of nodes, one or more intermediate layers, and an output layer of nodes. Connections between nodes may be created via the process of “training” the network, in which elements from a training data 304 set are applied to the input nodes, a suitable training algorithm (such as Levenberg-Marquardt, conjugate gradient, simulated annealing, or other algorithms) is then used to adjust the connections and weights between nodes in adjacent layers of the neural network to produce the desired values at the output nodes. This process is sometimes referred to as deep learning.

Still referring to FIG. 3, machine-learning algorithms may include at least a supervised machine-learning process 328. At least a supervised machine-learning process 328, as defined herein, include algorithms that receive a training set relating a number of inputs to a number of outputs, and seek to find one or more mathematical relations relating inputs to outputs, where each of the one or more mathematical relations is optimal according to some criterion specified to the algorithm using some scoring function. For instance, a supervised learning algorithm may include inputs and outputs as described above in this disclosure, and a scoring function representing a desired form of relationship to be detected between inputs and outputs; scoring function may, for instance, seek to maximize the probability that a given input and/or combination of elements inputs is associated with a given output to minimize the probability that a given input is not associated with a given output. Scoring function may be expressed as a risk function representing an “expected loss” of an algorithm relating inputs to outputs, where loss is computed as an error function representing a degree to which a prediction generated by the relation is incorrect when compared to a given input-output pair provided in training data 304. Persons skilled in the art, upon reviewing the entirety of this disclosure, will be aware of various possible variations of at least a supervised machine-learning process 328 that may be used to determine relation between inputs and outputs. Supervised machine-learning processes may include classification algorithms as defined above.

Further referring to FIG. 3, machine learning processes may include at least an unsupervised machine-learning processes 332. An unsupervised machine-learning process, as used herein, is a process that derives inferences in datasets without regard to labels; as a result, an unsupervised machine-learning process may be free to discover any structure, relationship, and/or correlation provided in the data. Unsupervised processes may not require a response variable; unsupervised processes may be used to find interesting patterns and/or inferences between variables, to determine a degree of correlation between two or more variables, or the like.

Still referring to FIG. 3, machine-learning module 300 may be designed and configured to create a machine-learning model 324 using techniques for development of linear regression models. Linear regression models may include ordinary least squares regression, which aims to minimize the square of the difference between predicted outcomes and actual outcomes according to an appropriate norm for measuring such a difference (e.g., a vector-space distance norm); coefficients of the resulting linear equation may be modified to improve minimization. Linear regression models may include ridge regression methods, where the function to be minimized includes the least-squares function plus term multiplying the square of each coefficient by a scalar amount to penalize large coefficients. Linear regression models may include least absolute shrinkage and selection operator (LASSO) models, in which ridge regression is combined with multiplying the least-squares term by a factor of 1 divided by double the number of samples. Linear regression models may include a multi-task lasso model wherein the norm applied in the least-squares term of the lasso model is the Frobenius norm amounting to the square root of the sum of squares of all terms. Linear regression models may include the elastic net model, a multi-task elastic net model, a least angle regression model, a LARS lasso model, an orthogonal matching pursuit model, a Bayesian regression model, a logistic regression model, a stochastic gradient descent model, a perceptron model, a passive aggressive algorithm, a robustness regression model, a Huber regression model, or any other suitable model that may occur to persons skilled in the art upon reviewing the entirety of this disclosure. Linear regression models may be generalized in an embodiment to polynomial regression models, whereby a polynomial equation (e.g. a quadratic, cubic or higher-order equation) providing a best predicted output/actual output fit is sought; similar methods to those described above may be applied to minimize error functions, as will be apparent to persons skilled in the art upon reviewing the entirety of this disclosure.

Continuing to refer to FIG. 3, machine-learning algorithms may include, without limitation, linear discriminant analysis. Machine-learning algorithm may include quadratic discriminate analysis. Machine-learning algorithms may include kernel ridge regression. Machine-learning algorithms may include support vector machines, including without limitation support vector classification-based regression processes. Machine-learning algorithms may include stochastic gradient descent algorithms, including classification and regression algorithms based on stochastic gradient descent. Machine-learning algorithms may include nearest neighbors' algorithms. Machine-learning algorithms may include various forms of latent space regularization such as variational regularization. Machine-learning algorithms may include Gaussian processes such as Gaussian Process Regression. Machine-learning algorithms may include cross-decomposition algorithms, including partial least squares and/or canonical correlation analysis. Machine-learning algorithms may include naïve Bayes methods. Machine-learning algorithms may include algorithms based on decision trees, such as decision tree classification or regression algorithms. Machine-learning algorithms may include ensemble methods such as bagging meta-estimator, forest of randomized tress, AdaBoost, gradient tree boosting, and/or voting classifier methods. Machine-learning algorithms may include neural net algorithms, including convolutional neural net processes.

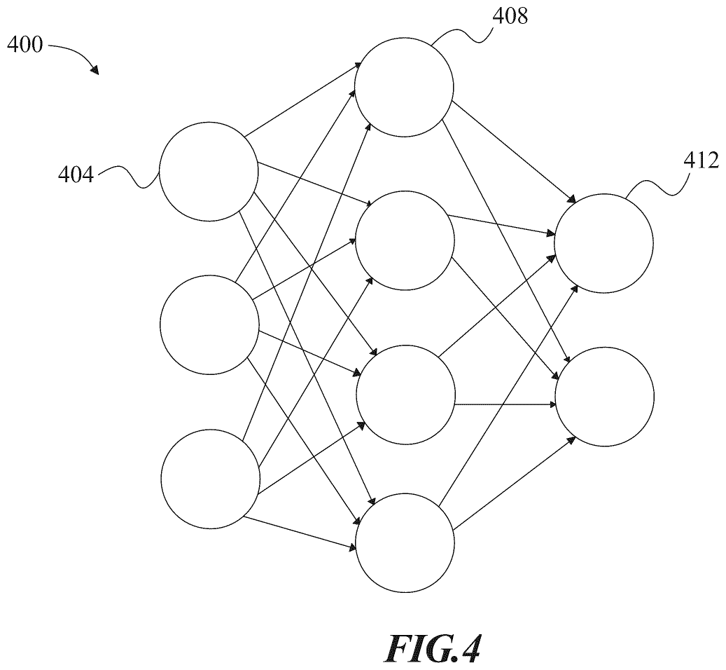

Referring now to FIG. 4, an exemplary embodiment of neural network 400 is illustrated. A neural network 400 also known as an artificial neural network, is a network of “nodes,” or data structures having one or more inputs, one or more outputs, and a function determining outputs based on inputs. Such nodes may be organized in a network, such as without limitation a convolutional neural network, including an input layer of nodes 404, one or more intermediate layers 408, and an output layer of nodes 412. Connections between nodes may be created via the process of “training” the network, in which elements from a training dataset are applied to the input nodes, a suitable training algorithm (such as Levenberg-Marquardt, conjugate gradient, simulated annealing, or other algorithms) is then used to adjust the connections and weights between nodes in adjacent layers of the neural network to produce the desired values at the output nodes. This process is sometimes referred to as deep learning. Connections may run solely from input nodes toward output nodes in a “feed-forward” network or may feed outputs of one layer back to inputs of the same or a different layer in a “recurrent network.” As a further non-limiting example, a neural network may include a convolutional neural network comprising an input layer of nodes, one or more intermediate layers, and an output layer of nodes. A “convolutional neural network,” as used in this disclosure, is a neural network in which at least one hidden layer is a convolutional layer that convolves inputs to that layer with a subset of inputs known as a “kernel,” along with one or more additional layers such as pooling layers, fully connected layers, and the like.

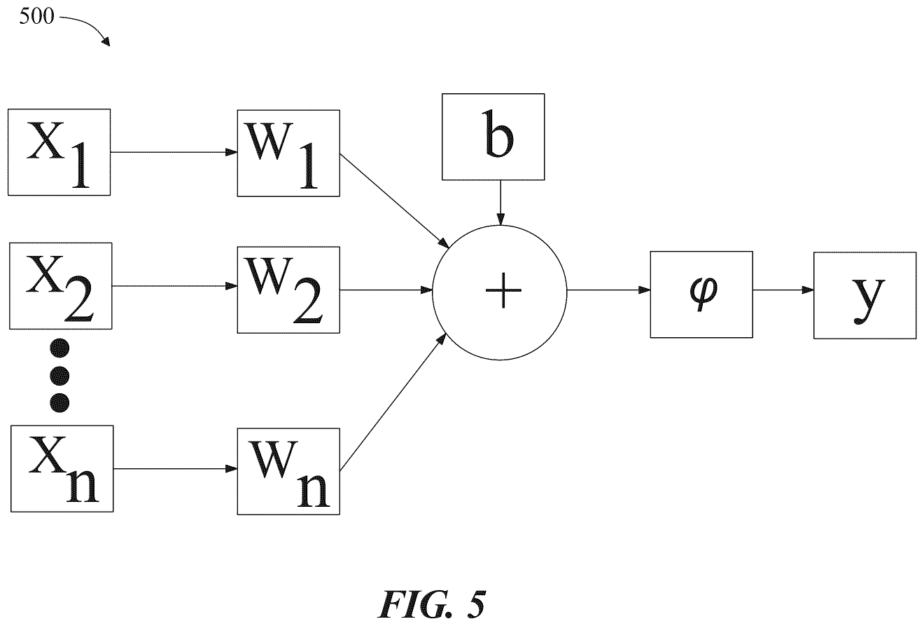

Referring now to FIG. 5, an exemplary embodiment 500 of a node of a neural network is illustrated. A node may include, without limitation a plurality of inputs xᵢ that may receive numerical values from inputs to a neural network containing the node and/or from other nodes. Node may perform a weighted sum of inputs using weights wᵢ that are multiplied by respective inputs xᵢ. Additionally or alternatively, a bias b may be added to the weighted sum of the inputs such that an offset is added to each unit in the neural network layer that is independent of the input to the layer. The weighted sum may then be input into a function φ, which may generate one or more outputs y. Weight wᵢ applied to an input xᵢ may indicate whether the input is “excitatory,” indicating that it has strong influence on the one or more outputs y, for instance by the corresponding weight having a large numerical value, and/or a “inhibitory,” indicating it has a weak effect influence on the one more inputs y, for instance by the corresponding weight having a small numerical value. The values of weights wᵢ may be determined by training a neural network using training data, which may be performed using any suitable process as described above.

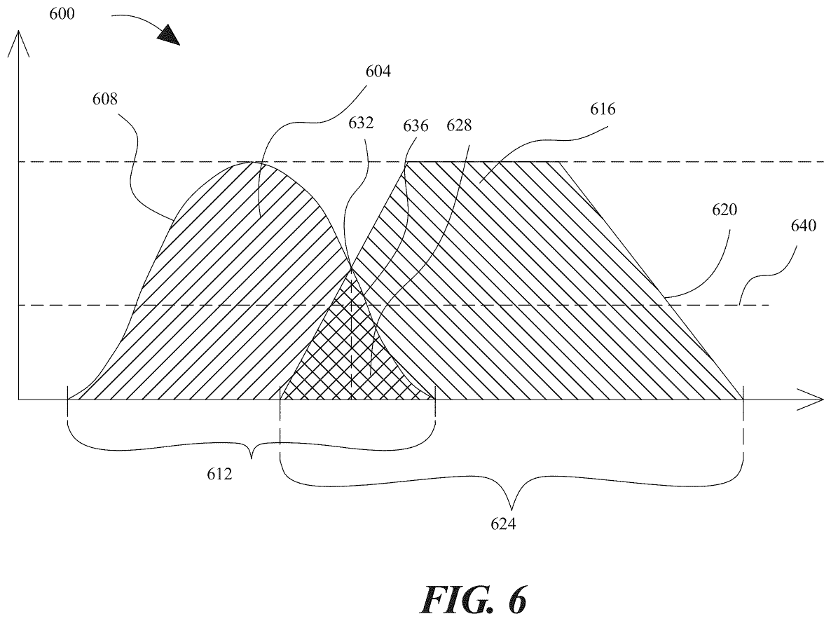

Referring to FIG. 6, an exemplary embodiment of fuzzy set comparison 600 is illustrated. This system may be implemented by inputting multiple potentially subjective determinations related to constraints which are represented as fuzzy sets and get output a probability distribution indicating likelihood that the compliance will be under the threshold, a degree to which it is over or under or a yes/no determination. A first fuzzy set 604 may be represented, without limitation, according to a first membership function 608 representing a probability that an input falling on a first range of values 612 is a member of the first fuzzy set 604, where the first membership function 608 has values on a range of probabilities such as without limitation the interval [0,1], and an area beneath the first membership function 608 may represent a set of values within first fuzzy set 604. Although first range of values 612 is illustrated for clarity in this exemplary depiction as a range on a single number line or axis, first range of values 612 may be defined on two or more dimensions, representing, for instance, a Cartesian product between a plurality of ranges, curves, axes, spaces, dimensions, or the like. First membership function 608 may include any suitable function mapping first range 612 to a probability interval, including without limitation a triangular function defined by two linear elements such as line segments or planes that intersect at or below the top of the probability interval. As a non-limiting example, triangular membership function may be defined as:

$$y ( x , a , b , c ) = \{ \begin{matrix}0 , for x > c and x < a \\ \frac{x - a}{b - a} , for a \leq x < b \\ \frac{c - x}{c - b} , if b < x \leq c\end{matrix}$$

a trapezoidal membership function may be defined as:

$$y ( a , b , c , d ) = max ( min ( \frac{x - a}{b - a} , 1 , \frac{d - x}{d - c} ) , 0 )$$

a sigmoidal function may be defined as:

$$y ( x , a , c ) = \frac{1}{1 - {e}^{- ( x - c )}}$$

a Gaussian membership function may be defined as:

$$y ( x , c , σ ) = {e}^{- \frac{1}{2} {( \frac{x - c}{σ} )}^{2}}$$

and a bell membership function may be defined as:

$$y ( x , a , b , c , ) = {[ 1 + {\vert \frac{x - c}{a} \vert}^{2 b} ]}^{- 1}$$

Persons skilled in the art, upon reviewing the entirety of this disclosure, will be aware of various alternative or additional membership functions that may be used consistently with this disclosure.

Still referring to FIG. 6, first fuzzy set 604 may represent any value or combination of values as described above, including output from one or more machine-learning models. A second fuzzy set 616, which may represent any value which may be represented by first fuzzy set 604, may be defined by a second membership function 620 on a second range 624; second range 624 may be identical and/or overlap with first range 612 and/or may be combined with first range via Cartesian product or the like to generate a mapping permitting evaluation overlap of first fuzzy set 604 and second fuzzy set 616. Where first fuzzy set 604 and second fuzzy set 616 have a region 628 that overlaps, first membership function 608 and second membership function 620 may intersect at a point 632 representing a probability, as defined on probability interval, of a match between first fuzzy set 604 and second fuzzy set 616. Alternatively or additionally, a single value of first and/or second fuzzy set may be located at a locus 636 on first range 612 and/or second range 624, where a probability of membership may be taken by evaluation of first membership function 608 and/or second membership function 620 at that range point. A probability at 628 and/or 632 may be compared to a threshold 640 to determine whether a positive match is indicated. Threshold 640 may, in a non-limiting example, represent a degree of match between first fuzzy set 604 and second fuzzy set 616, and/or single values therein with each other or with either set, which is sufficient for purposes of the matching process; for instance, threshold may indicate a sufficient degree of overlap between an output from one or more machine-learning models and a predetermined class, for combination to occur as described above. Alternatively or additionally, each threshold may be tuned by a machine-learning and/or statistical process, for instance and without limitation as described in further detail below.

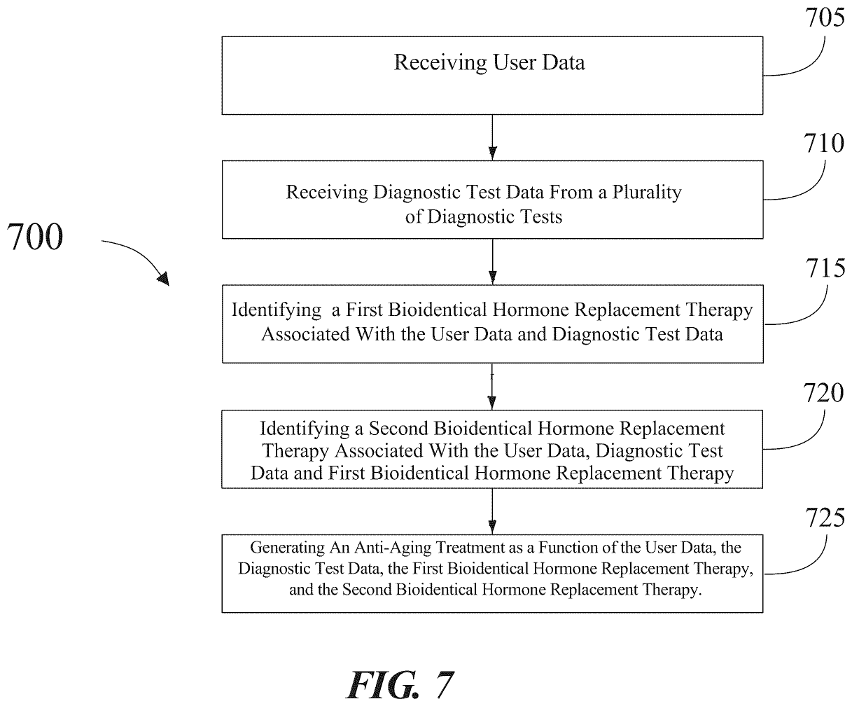

Referring now to FIG. 7, a method 700 of using an apparatus for an anti-aging treatment is illustrated. At step 705, method 700 includes receiving user data 120. User data 120 may be received through user input, from external computing devices, such as a remote device, and the like. This step may be implemented as described above in FIGS. 1-6, without limitation.

Still referring to FIG. 7, at step 710, method 700 includes receiving diagnostic test data 124 from a plurality of diagnostic tests. This step may be implemented as described above in FIGS. 1-6, without limitation.

Still referring to FIG. 7, at step 715, method 700 includes identifying a first bioidentical hormone replacement therapy 136 associated with user data 120 and diagnostic test data 124. This step may be implemented as described above in FIGS. 1-6, without limitation.

Still referring to FIG. 7, at step 720, method 700 includes identifying second bioidentical hormone replacement therapy 140 associated with user data 120, diagnostic test data 124 and first bioidentical hormone replacement therapy 136. This step may be implemented as described above in FIGS. 1-6, without limitation.

Still referring to FIG. 7, at step 725, method 700 includes generating an anti-aging treatment 144 as a function of user data 120, diagnostic test data 124, first bioidentical hormone replacement therapy 136 and second bioidentical hormone replacement therapy 140. This step may be implemented as described above in FIGS. 1-6, without limitation.

It is to be noted that any one or more of the aspects and embodiments described herein may be conveniently implemented using one or more machines (e.g., one or more computing devices that are utilized as a user computing device for an electronic document, one or more server devices, such as a document server, etc.) programmed according to the teachings of the present specification, as will be apparent to those of ordinary skill in the computer art. Appropriate software coding can readily be prepared by skilled programmers based on the teachings of the present disclosure, as will be apparent to those of ordinary skill in the software art. Aspects and implementations discussed above employing software and/or software modules may also include appropriate hardware for assisting in the implementation of the machine executable instructions of the software and/or software module.

Such software may be a computer program product that employs a machine-readable storage medium. A machine-readable storage medium may be any medium that is capable of storing and/or encoding a sequence of instructions for execution by a machine (e.g., a computing device) and that causes the machine to perform any one of the methodologies and/or embodiments described herein. Examples of a machine-readable storage medium include, but are not limited to, a magnetic disk, an optical disc (e.g., CD, CD-R, DVD, DVD-R, etc.), a magneto-optical disk, a read-only memory “ROM” device, a random access memory “RAM” device, a magnetic card, an optical card, a solid-state memory device, an EPROM, an EEPROM, and any combinations thereof. A machine-readable medium, as used herein, is intended to include a single medium as well as a collection of physically separate media, such as, for example, a collection of compact discs or one or more hard disk drives in combination with a computer memory. As used herein, a machine-readable storage medium does not include transitory forms of signal transmission.

Such software may also include information (e.g., data) carried as a data signal on a data carrier, such as a carrier wave. For example, machine-executable information may be included as a data-carrying signal embodied in a data carrier in which the signal encodes a sequence of instruction, or portion thereof, for execution by a machine (e.g., a computing device) and any related information (e.g., data structures and data) that causes the machine to perform any one of the methodologies and/or embodiments described herein.

Examples of a computing device include, but are not limited to, an electronic book reading device, a computer workstation, a terminal computer, a server computer, a handheld device (e.g., a tablet computer, a smartphone, etc.), a web appliance, a network router, a network switch, a network bridge, any machine capable of executing a sequence of instructions that specify an action to be taken by that machine, and any combinations thereof. In one example, a computing device may include and/or be included in a kiosk.

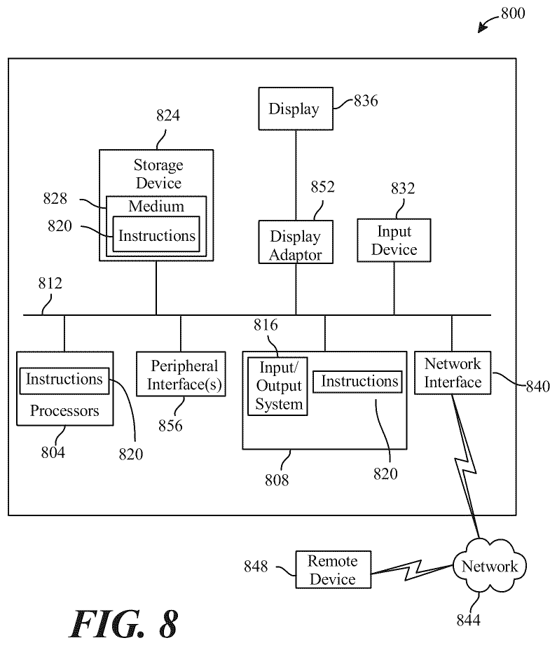

FIG. 8 shows a diagrammatic representation of one embodiment of a computing device in the exemplary form of a computer system 800 within which a set of instructions for causing a control system to perform any one or more of the aspects and/or methodologies of the present disclosure may be executed. It is also contemplated that multiple computing devices may be utilized to implement a specially configured set of instructions for causing one or more of the devices to perform any one or more of the aspects and/or methodologies of the present disclosure. Computer system 800 includes a processor 804 and a memory 808 that communicate with each other, and with other components, via a bus 812. Bus 812 may include any of several types of bus structures including, but not limited to, a memory bus, a memory controller, a peripheral bus, a local bus, and any combinations thereof, using any of a variety of bus architectures.

Processor 804 may include any suitable processor, such as without limitation a processor incorporating logical circuitry for performing arithmetic and logical operations, such as an arithmetic and logic unit (ALU), which may be regulated with a state machine and directed by operational inputs from memory and/or sensors; processor 804 may be organized according to Von Neumann and/or Harvard architecture as a non-limiting example. Processor 804 may include, incorporate, and/or be incorporated in, without limitation, a microcontroller, microprocessor, digital signal processor (DSP), Field Programmable Gate Array (FPGA), Complex Programmable Logic Device (CPLD), Graphical Processing Unit (GPU), general purpose GPU, Tensor Processing Unit (TPU), analog or mixed signal processor, Trusted Platform Module (TPM), a floating-point unit (FPU), and/or system on a chip (SoC).

Memory 808 may include various components (e.g., machine-readable media) including, but not limited to, a random-access memory component, a read only component, and any combinations thereof. In one example, a basic input/output system 816 (BIOS), including basic routines that help to transfer information between elements within computer system 800, such as during start-up, may be stored in memory 808. Memory 808 may also include (e.g., stored on one or more machine-readable media) instructions (e.g., software) 820 embodying any one or more of the aspects and/or methodologies of the present disclosure. In another example, memory 808 may further include any number of program modules including, but not limited to, an operating system, one or more application programs, other program modules, program data, and any combinations thereof.

Computer system 800 may also include a storage device 824. Examples of a storage device (e.g., storage device 824) include, but are not limited to, a hard disk drive, a magnetic disk drive, an optical disc drive in combination with an optical medium, a solid-state memory device, and any combinations thereof. Storage device 824 may be connected to bus 812 by an appropriate interface (not shown). Example interfaces include, but are not limited to, SCSI, advanced technology attachment (ATA), serial ATA, universal serial bus (USB), IEEE 1394 (FIREWIRE), and any combinations thereof. In one example, storage device 824 (or one or more components hereof) may be removably interfaced with computer system 800 (e.g., via an external port connector (not shown)). Particularly, storage device 824 and an associated machine-readable medium 828 may provide nonvolatile and/or volatile storage of machine-readable instructions, data structures, program modules, and/or other data for computer system 800. In one example, software 820 may reside, completely or partially, within machine-readable medium 828. In another example, software 820 may reside, completely or partially, within processor 804.

Computer system 800 may also include an input device 832. In one example, a user of computer system 800 may enter commands and/or other information into computer system 800 via input device 832. Examples of an input device 832 include, but are not limited to, an alpha-numeric input device (e.g., a keyboard), a pointing device, a joystick, a gamepad, an audio input device (e.g., a microphone, a voice response system, etc.), a cursor control device (e.g., a mouse), a touchpad, an optical scanner, a video capture device (e.g., a still camera, a video camera), a touchscreen, and any combinations thereof. Input device 832 may be interfaced to bus 812 via any of a variety of interfaces (not shown) including, but not limited to, a serial interface, a parallel interface, a game port, a USB interface, a FIREWIRE interface, a direct interface to bus 812, and any combinations thereof. Input device 832 may include a touch screen interface that may be a part of or separate from display 836, discussed further below. Input device 832 may be utilized as a user selection device for selecting one or more graphical representations in a graphical interface as described above.

A user may also input commands and/or other information to computer system 800 via storage device 824 (e.g., a removable disk drive, a flash drive, etc.) and/or network interface device 840. A network interface device, such as network interface device 840, may be utilized for connecting computer system 800 to one or more of a variety of networks, such as network 844, and one or more remote devices 848 connected thereto. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network, such as network 844, may employ a wired and/or a wireless mode of communication. In general, any network topology may be used. Information (e.g., data, software 820, etc.) may be communicated to and/or from computer system 800 via network interface device 840.

Computer system 800 may further include a video display adapter 852 for communicating a displayable image to a display device, such as display device 836. Examples of a display device include, but are not limited to, a liquid crystal display (LCD), a cathode ray tube (CRT), a plasma display, a light emitting diode (LED) display, and any combinations thereof. Display adapter 852 and display device 836 may be utilized in combination with processor 804 to provide graphical representations of aspects of the present disclosure. In addition to a display device, computer system 800 may include one or more other peripheral output devices including, but not limited to, an audio speaker, a printer, and any combinations thereof. Such peripheral output devices may be connected to bus 812 via a peripheral interface 856. Examples of a peripheral interface include, but are not limited to, a serial port, a USB connection, a FIREWIRE connection, a parallel connection, and any combinations thereof.

The foregoing has been a detailed description of illustrative embodiments of the invention. Various modifications and additions can be made without departing from the spirit and scope of this invention. Features of each of the various embodiments described above may be combined with features of other described embodiments as appropriate in order to provide a multiplicity of feature combinations in associated new embodiments. Furthermore, while the foregoing describes a number of separate embodiments, what has been described herein is merely illustrative of the application of the principles of the present invention. Additionally, although particular methods herein may be illustrated and/or described as being performed in a specific order, the ordering is highly variable within ordinary skill to achieve methods and systems according to the present disclosure. Accordingly, this description is meant to be taken only by way of example, and not to otherwise limit the scope of this invention.

Exemplary embodiments have been disclosed above and illustrated in the accompanying drawings. It will be understood by those skilled in the art that various changes, omissions and additions may be made to that which is specifically disclosed herein without departing from the spirit and scope of the present invention.

1. An apparatus for an anti-aging treatment, the apparatus comprising:
at least a processor; and
a memory communicatively connected to the at least processor, the memory containing instructions configuring the at least a processor to:
receive user data of a user comprising user stress level and severity of symptoms;
receive diagnostic test data from a plurality of diagnostic tests;
identify a first bioidentical hormone replacement therapy (BHRT) as a function of the user data and the diagnostic test data, wherein the first BHRT comprises:
a first customized treatment program generated using a machine-learning module trained using training data, wherein the machine-learning module is generated by creating an artificial neural network which further comprises:
receiving the training data, wherein the training data comprises a plurality of user data entries as inputs and a plurality of correlated customized treatment programs as outputs;
training, iteratively, the machine-learning module using the training data, wherein training the machine-learning module includes retraining the machine-learning module with feedback from previous iterations of the machine-learning module;
identify a second BHRT associated with the first BHRT based on a first user response from the first BHRT received through a graphical user interface (GUI) from the user, wherein identifying the second BHRT comprises:
identifying a titration by comparing the first user response from the first BHRT to a predetermined threshold;
modifying the first customized treatment program as a function of the identified titration;
retraining the machine-learning module using updated training data by including the modified first customized treatment program in the updated training data, wherein the updated training data includes previous outputs such that the retrained machine-learning module iteratively produces outputs, creating a feedback loop; and
generating a second customized treatment program using the retrained machine-learning module; and
generate an anti-aging treatment configured to update the diagnostic test data as a function of the second customized treatment program and a second user response from the second BHRT received through the GUI from the user.

2. The apparatus of claim 1, wherein the user data comprises user social data.

3. The apparatus of claim 1, wherein a diagnostic test of the plurality of diagnostic tests comprises an autoimmune test.

4. The apparatus of claim 1, wherein:
the plurality of diagnostic tests comprises a plurality of male user tests if the user is male; and
the plurality of diagnostic tests comprises a plurality of female user tests if the user is female.

5. The apparatus of claim 1, wherein the first bioidentical hormone replacement therapy comprises topical optimized hormones.

6. (canceled)

7. (canceled)

8. The apparatus of claim 1, wherein the second bioidentical hormone replacement therapy comprises a target.

9. The apparatus of claim 8, wherein the second bioidentical hormone replacement therapy comprises customized peptides for the target.

10. The apparatus of claim 1, wherein generating the anti-aging treatment comprises using updated diagnostic test data after the at least the first bioidentical hormone replacement therapy.

11. A method for an anti-aging treatment, the method comprising:
receiving, by at least a processor, user data of a user comprising user stress level and severity of symptoms;
receiving, by the at least a processor, diagnostic test data from a plurality of diagnostic tests;
identifying, by the at least a processor, a first bioidentical hormone replacement therapy (BHRT) as a function of the user data and diagnostic test data, wherein the first BHRT comprises:
a first customized treatment program generated using a machine-learning module trained using training data, wherein the machine-learning module is generated by creating an artificial neural network, which further comprises:
receiving the training data, wherein the training data comprises a plurality of user data entries as inputs and a plurality of correlated customized treatment programs as outputs, and wherein the training data is mapped to one or more descriptors of categories;
training, iteratively, the machine-learning module using the training data, wherein training the machine-learning module includes retraining the machine-learning module with feedback from previous iterations of the machine-learning module;
identifying, by the at least a processor, a second BHRT associated with the first BHRT based on a first response from the first BHRT received through a graphical user interface (GUI) from the user, wherein identifying the second BHRT comprises:
identifying a titration by comparing the first user response from the first BHRT to a predetermined threshold;
modifying the first customized treatment program as a function of the identified titration;
retraining the machine-learning module using updated training data by including the modified first customized treatment program in the updated training data, wherein the updated training data includes previous outputs such that the retrained machine-learning module iteratively produces outputs, creating a feedback loop; and
generating a second customized treatment program using the retrained machine-learning module;
generating, by the at least a processor, an anti-aging treatment configured to update the diagnostic test data as a function of the second customized treatment program and a second user response from the second BHRT received through the GUI from the user.

12. The method of claim 11, wherein the user data comprises user social data.

13. The method of claim 11, wherein a diagnostic test of the plurality of diagnostic tests comprises an autoimmune test.

14. The method of claim 11, wherein:
the plurality of diagnostic tests comprises a plurality of male user tests if the user is male; and
the plurality of diagnostic tests comprises a plurality of female user tests if the user is female.

15. The method of claim 11, wherein the first bioidentical hormone replacement therapy comprises topical optimized hormones.

16. (canceled)

17. (canceled)

18. The method of claim 11, wherein the second bioidentical hormone replacement therapy comprises a target.

19. The method of claim 18, wherein the second bioidentical hormone replacement therapy comprises customized peptides for the target.

20. The method of claim 11, wherein generating the anti-aging treatment comprises using updated diagnostic test data after the at least the first bioidentical hormone replacement therapy.
